(19) United States
(12) Patent Application Publication
(10) Pub. No.: US 2021/0236696 A1
(43) Pub. Date: Aug. 5, 2021

(54) Artificial nerve conduit construction using tissue engineering methods

(71) Applicant: Jiangnan University, Wuxi (CN)

(72) Inventors: Naiyan LU, Wuxi (CN); Xuan ZHANG, Wuxi (CN); Yuyan WENG, Wuxi (CN); Xuejian YU, Wuxi (CN); Guofeng YANG, Wuxi (CN)

(21) Appl. No.: 17/228,089

(22) Filed: Apr. 12, 2021

Related U.S. Application Data
(63) Continuation of application No. PCT/CN2019/010364, filed on Aug. 30, 2019.

(51) Int. Cl. A61L 27/38; A61L 27/20; A61L 27/22; A61L 27/54; A61L 27/36; A61L 27/16; A61L 27/18; G03F 7/00; G03F 7/20; G03F 7/16

(52) U.S. Cl. CPC A61L 27/3834; A61L 2300/64; A61L 27/225; A61L 27/54; A61L 27/3895; A61L 27/3675; A61L 27/16; A61L 27/18; A61L 27/383; A61L 27/3886; G03F 7/0002; G03F 7/70008; G03F 7/162; A61L 2300/414; A61L 2430/32; A61L 27/20

(57) ABSTRACT

The disclosure discloses a tissue-engineered nerve transplant and a preparation method thereof, and belongs to the technical fields of biomaterials and tissue engineering. By optimizing the specification of stripes, the stripes can independently induce EMSCs to differentiate to myelination cells (Schwann cells) to the maximum extent so as to obtain an EMSCs/biomaterial scaffold compound. The EMSCs/biomaterial scaffold compound can not only be used as a three-dimensional cell culture model for researching neural stem cell differentiation, nerve fiber growth and myelination molecular mechanisms in vitro, but also be used as a tissue engineering transplant for in-vivo transplantation to repair nervous system injury. In the disclosure, an EMSCs/micropatterned biomaterial film is rolled into a cylindrical multi-tunnel type nerve regeneration conduit to be used to repair sciatic nerve injury by transplantation, and results show that the disclosure can promote nerve regeneration and recovery of a lower limb motor function through injured portion transplantation, and has good clinical application prospects and research and development value.

### TECHNICAL FIELD

The disclosure relates to a tissue-engineered nerve transplant and a preparation method thereof, and belongs to the technical fields of biomaterials and tissue engineering.

### BACKGROUND

Stem cell/tissue engineering scaffold transplantation is a main strategy for repairing nervous tissue damage. In the central nervous system, nerve cells of the brain form a network structure by mutual connection of neurites. After brain tissue damage, the main purpose of stem cell/scaffold transplantation is to promote formation of a new neural network to recover its information integration and conduction functions, and therefore planted stem cells/scaffolds are required to promote formation of the neural network; and compared with the information integration function of the brain tissue, the spinal cord has the main function of conducting brain information to motor neurons of the spinal cord via descending conduction bundles such as the corticospinal tract and uploading sensory information received by the spinal cord to the brain via ascending sensory conduction bundles such as the fasciculus gracilis and the fasciculus cuneatus. Therefore, after spinal cord damage, stem cell/tissue engineering scaffold transplantation is mainly to promote regeneration of nervous conduction bundles and parallel growth of nerve fibers so as to make the nerve fibers pass damaged portions forwards and directionally extend to spinal cord tissue at the damage far ends (motor nerves) or near ends (sensory nerves), and the regenerated nerve fibers grow along an original passage and finally have synapse connection with their target cells again. In the meantime, the transplanted stem cells/tissue engineering scaffolds further should have the effect of promoting new nerve fibers to form myelin sheaths. A peripheral nerve injury repairing mechanism is similar to a spinal cord injury repairing mechanism, and repairing peripheral nerves by stem cell/tissue engineering scaffold (conduit) transplantation, e.g., sciatic nerve injury, also promotes parallel effective regeneration and myelination of the nerve fibers. Therefore, the key factor of improving the treatment effect of repairing spinal cord or peripheral nerve injury by stem cell/tissue engineering scaffold transplantation is how to physically and chemically modify the scaffolds and select proper seed cells (having the capability of differentiating into myelination cells) to be planted to the scaffolds to induce parallel growth and myelination of the nerve fibers.

At present, repairing spinal cord and peripheral nerve (e.g., sciatic nerve) injury by stem cell/scaffold transplantation is mainly to manufacture hydrogel or nerve conduits from stem cell/biological materials and transplant them to injured and defected portions. Especially when traumas cause longer defects of peripheral nerves, nerve conduit transplantation will be the most effective treatment method for repairing nerve defects. Although most of the nerve conduits used at present can promote nerve regeneration, due to the lack of stripes guiding the nerve fibers to grow in parallel and orderly, the direction of nerve fiber extension is lost, which makes the fibers move forwards slowly. At the same time, due to the lack of myelination cells, the myelination of the regenerated nerve fibers is incomplete (myelin sheaths are formed only relying on proliferation of residual endogenous Schwann cells at the nerve broken ends). Therefore, it is urgent to invent and develop a nerve conduit that can not only obviously promote the parallel directional growth of the regenerated nerve fibers, but also provide myelination seed cells and promote the regeneration and myelination of injured nerves in vivo, for transplantation so as to repair nerve injury more effectively.

### SUMMARY

The disclosure provides a tissue-engineered nerve transplant, wherein a biomaterial with a surface provided with a striped micropattern is used as a scaffold, ecto-mesenchymal stem cells (EMSCs) are used as seed cells, and the scaffold is inoculated with the seed cells to form the tissue-engineered nerve transplant.

In an embodiment, the surface of the biomaterial has the striped micropattern, and the striped micropattern is imprinted through a micropattern technology which includes, but is not limited to, photoetching, electron beam lithography or nanoimprint lithography.

In an embodiment, the striped micropattern has a width of 1-2 μm, a spacing of 1-2 μm and a stripe height of 1-2 μm.

In an embodiment, one or more of polydimethylsiloxane (PDMS), polycaprolactone (PCL), chitosan and fibrinogen are used as the biomaterial.

In an embodiment, the biomaterial includes chitosan-fibrous protein.

In an embodiment, the chitosan-fibrous protein is obtained by crosslinking chitosan and fibrinogen with a cell growth factor through a biological crosslinking agent, and the cell growth factor is one or more of epidermal growth factor(EGF), fibroblast growth factor (FGE), nerve growth factor (NGF) and sonic hedgehog homolog (SHH).

In an embodiment, the biological crosslinking agent includes genipin or/and glutamine transaminase (TG).

In an embodiment, an initial cell density of the EMSCs is 10⁴-10⁵ cells/cm².

In an embodiment, the tissue-engineered nerve transplant has a shape including a film shape.

In an embodiment, the tissue-engineered nerve transplant is rolled into a single-layer or multi-layer multi-tunnel nerve conduit.

In an embodiment, the tissue-engineered nerve transplant is filled with a drug or growth factor sustained release material for promoting nerve growth.

In an embodiment, the drug or growth factor sustained release material for promoting nerve growth includes a drug sustained release system with microspheres, nanoparticles or hydrogel as a carrier.

In an embodiment, the tissue-engineered nerve transplant is configured to repair nerve injury.

The disclosure further provides a method for preparing the above tissue-engineered nerve transplant. The method includes the following steps:

(1) preparing a biomaterial scaffold with a surface provided with a micropattern;

(2) performing material-taking, culture and amplification of EMSCs; and

(3) planting the EMSCs prepared in step (2) to the biomaterial scaffold with the surface provided with the micropattern in step (1).

The disclosure further provides a nerve conduit. A biomaterial with a surface provided with a striped micropattern is used as a scaffold, ecto-mesenchymal stem cells (EMSCs) are used as seed cells, the scaffold is inoculated with the seed cells to obtain a tissue-engineered nerve transplant, and the tissue-engineered nerve transplant is rolled into the single-layer or multi-layer multi-tunnel nerve conduit.

In an embodiment, the striped micropattern has a width of 1-2 μm, a spacing of 1-2 μm and a stripe height of 1-2 μm.

In an embodiment, one or more of polydimethylsiloxane, polycaprolactone, chitosan and fibrinogen are used as the biomaterial.

The disclosure further provides application of the above tissue-engineered nerve transplant or the above nerve conduit in preparation of medical instruments.

### Beneficial Effects

In the disclosure, an EMSCs/micropatterned biomaterial film is rolled into a cylindrical multi-tunnel nerve regeneration conduit which is configured to repair sciatic nerve injury by transplantation. Results show that a sciatic nerve function index of a sciatic nerve injury side of a mouse without nerve conduit treatment reaches −91±25, while a sciatic nerve function index of a sciatic nerve injury side of a mouse treated through the nerve conduit of the application reaches −37±17. The tissue-engineered nerve transplant provided by the disclosure can promote nerve regeneration and recovery of a lower limb motor function through injured portion transplantation, and has good clinical application prospects and research and development value.

### BRIEF DESCRIPTION OF FIGURES

FIG. 1A is a surface-striped micropattern PDMS film with the stripe specification of 0.5 μm×0.5 μm×0.5 μm.

FIG. 1B is a surface-striped micropattern PDMS film with the stripe specification of 1.0 μm×1.0 μm×1.0 μm.

FIG. 1C is a surface-striped micropattern PDMS film with the stripe specification of 1.5 μm×1.5 μm×1.5 μm.

FIG. 1D is a surface-striped micropattern PDMS film with the stripe specification of 2.0 μm×2.0 μm×2.0 μm.

FIG. 2A is a fluorescent staining (S100) image (shot via a fluorescence microscope) after differentiating to Schwann cells from EMSCs planted on a surface of a micropatterned PDMS film with the stripe specification of 0.5 μm×0.5 μm×0.5 μm.

FIG. 2B is a fluorescent staining (S100) image (shot via a fluorescence microscope) after differentiating to Schwann cells from EMSCs planted on a surface of a micropatterned PDMS film with the stripe specification of 1.0 μm×1.0 μm×1.0 μm.

FIG. 2C is a fluorescent staining (S100) image (shot via a fluorescence microscope) after differentiating to Schwann cells from EMSCs planted on a surface of a micropatterned PDMS film with the stripe specification of 1.5 μm×1.5 μm×1.5 μm.

FIG. 2D is a fluorescent staining (S100) image (shot via a fluorescence microscope) after differentiating to Schwann cells from EMSCs planted on a surface of a micropatterned PDMS film with the stripe specification of 2.0 μm×2.0 μm×2.0 μm.

FIG. 3 shows Western blotting detection results of levels of cells expressing Schwann cell marker proteins after differentiating to Schwann cells from EMSCs planted on a surface of a micropatterned PDMS film; 1, Non-stripe film; 2, 0.5 μm stripe; 3, 1 μm stripe; 4, 1.5 μm stripe; 5, 2 μm stripe; 6, 2.5 μm stripe.

FIG. 4A is a state diagram of radial growth of a neural stem cell on a surface of a non-stripe PCL film.

FIG. 4B is a state diagram of parallel growth of nerve fibers of a neural stem cell on a surface of a striped (1.0 μm×1.0 μm×1.0 μm) PCL film.

FIG. 4C is a state diagram of growth, along a stripe, of a nerve fiber of a neural stem cell on a surface of a (1.0 μm×1.0 μm×1.0 μm) PCL film.

FIG. 5A shows a growth condition of a nerve cell on a surface of an EMSCs (Schwann cells)/striped micropattern PCL film (1.0 μm×1.0 μm×1.0 μm).

FIG. 5B shows a growth condition of a nerve cell on a surface of an EMSCs (Schwann cells)/striped micropattern PCL film (2.0 μm×2.0 μm×2.0 μm).

FIG. 6 is a schematic diagram of an EMSCs (Schwann cells)/striped micropattern film (conduit) transplantation operation on a sciatic nerve injury rat animal model.

FIG. 7A is a state diagram of cutoff of a sciatic nerve of a rat animal model.

FIG. 7B shows that nerve broken ends (two ends are connected and aligned through an absorbable suture, while a 5 mm spacing is reserved in the middle) are wrapped with an EMSCs (Schwann cells)/striped (1.0 μm×1.0 μm×1.0 μm) micropattern PCL composite film (the film is deep blue due to crosslinking through genipin) and sealed through fibrin glue.

FIG. 7C is a treatment state diagram of rolling an EMSCs (Schwann cells)/striped (1.0 μm×1.0 μm×1.0 μm) micropattern PCL composite film into a conduit, suturing the conduit through an absorbable suture and then sealing an outer surface of the conduit through fibrin glue.

FIG. 8A is a state diagram of separating and cutting off a sciatic nerve of a rat animal model.

FIG. 8B is a treatment state diagram of an operation process that nerve broken ends of a rat sciatic nerve injury animal model are wrapped with an EMSCs (Schwann cells)/striped (1.0 μm×1.0 μm×1.0 μm) micropattern fibrous protein/chitosan composite film and then sealed through fibrin glue.

FIG. 8C is a treatment state diagram of rolling a micropattern fibrous protein/chitosan composite film into a conduit, suturing the conduit through an absorbable suture and then sealing an outer surface of the conduit through fibrin glue for a rat sciatic nerve injury animal model.

FIG. 9A shows a tracing result of nerve cells in a dorsal root ganglion of a mouse in a normal group with fluorochrome injection into a sciatic nerve.

FIG. 9B shows a tracing result of nerve cells in a dorsal root ganglion of a mouse in group 1, namely an injured non-transplanted with fluorochrome injection into a sciatic nerve.

FIG. 9C shows a tracing result of nerve cells in a dorsal root ganglion of a mouse in group 5 with fluorochrome injection into a sciatic nerve.

FIG. 9D shows a tracing result of nerve cells in a dorsal root ganglion of a mouse in group 4 with fluorochrome injection into a sciatic nerve.

FIG. 9E shows a tracing result of nerve cells in a dorsal root ganglion of a mouse in group 3 with fluorochrome injection into a sciatic nerve.

FIG. 9F shows a tracing result of nerve cells in a dorsal root ganglion of a mouse in group 2 with fluorochrome injection into a sciatic nerve.

FIG. 10A shows an appearance of gastrocnemius of a lower limb on a normal side.

FIG. 10B shows a general appearance of gastrocnemius on a sciatic nerve injury side of group 1, namely an injured non-transplanted group.

FIG. 10C shows a general appearance of gastrocnemius on a sciatic nerve injury side of group 5.

FIG. 10D shows a general appearance of gastrocnemius on a sciatic nerve injury side of group 4.

FIG. 10E shows a general appearance of gastrocnemius on a sciatic nerve injury side of group 3.

FIG. 10F shows a general appearance of gastrocnemius on a sciatic nerve injury side of group 2.

FIG. 11A is a cross-sectional view of gastrocnemius fibers after HE staining on a tissue slice of gastrocnemius of a lower limb on a normal side.

FIG. 11B is a cross-sectional view of gastrocnemius fibers after HE staining on a tissue slice of gastrocnemius on a sciatic nerve injury side of group 1, namely an injured non-transplanted group.

FIG. 11C is a cross-sectional view of gastrocnemius fibers after HE staining on a tissue slice of gastrocnemius on a sciatic nerve injury side of group 5, namely an injured non-transplanted group.

FIG. 11D is a cross-sectional view of gastrocnemius fibers after HE staining on a tissue slice of gastrocnemius on a sciatic nerve injury side of group 4, namely an injured non-transplanted group.

FIG. 11E is a cross-sectional view of gastrocnemius fibers after HE staining on a tissue slice of gastrocnemius on a sciatic nerve injury side of group 3, namely an injured non-transplanted group.

FIG. 11F is a cross-sectional view of gastrocnemius fibers after HE staining on a tissue slice of gastrocnemius on a sciatic nerve injury side of group 2, namely an injured non-transplanted group.

FIG. 12A shows a growth condition of a nerve in a conduit after steps that 16 weeks after an EMSCs (Schwann cells)/striped micropattern PCL composite film transplantation operation on a rat sciatic nerve injury animal model in group 5, a sciatic nerve is taken after the animal is anesthetized, a transported nerve conduit together with nerves at far and near ends are taken out and fixed, and the conduit is split along a longitudinal axis (an original conduit wall has been rebuilt by tissue in vivo).

FIG. 12B shows a growth condition of a nerve in a conduit after steps that 16 weeks after an EMSCs (Schwann cells)/striped micropattern PCL composite film transplantation operation on a rat sciatic nerve injury animal model in group 4, a sciatic nerve is taken after the animal is anesthetized, a transported nerve conduit together with nerves at far and near ends are taken out and fixed, and the conduit is split along a longitudinal axis (an original conduit wall has been rebuilt by tissue in vivo).

FIG. 12C shows a growth condition of a nerve in a conduit after steps that 16 weeks after an EMSCs (Schwann cells)/striped micropattern PCL composite film transplantation operation on a rat sciatic nerve injury animal model in group 3, a sciatic nerve is taken after the animal is anesthetized, a transported nerve conduit together with nerves at far and near ends are taken out and fixed, and the conduit is split along a longitudinal axis (an original conduit wall has been rebuilt by tissue in vivo).

FIG. 12D shows a growth condition of a nerve in a conduit after steps that 16 weeks after an EMSCs (Schwann cells)/striped micropattern PCL composite film transplantation operation on a rat sciatic nerve injury animal model in group 2, a sciatic nerve is taken after the animal is anesthetized, a transported nerve conduit together with nerves at far and near ends are taken out and fixed, and the conduit is split along a longitudinal axis (an original conduit wall has been rebuilt by tissue in vivo).

FIG. 13A shows an observation result of HE staining on a sciatic nerve tissue slice at an injured portion with a transplanted nerve conduit in group 5.

FIG. 13B shows an observation result of HE staining on a sciatic nerve tissue slice at an injured portion without a transplanted nerve conduit.

FIG. 14A shows a regeneration condition of a nerve fiber after immunohistochemical staining with a nerve fiber marker protein NF-200 on a longitudinal tissue slice of a sciatic nerve of a lower limb on a normal side.

FIG. 14B shows a regeneration condition of a nerve fiber after immunohistochemical staining with a nerve fiber marker protein NF-200 on a longitudinal tissue slice of a sciatic nerve injury portion (including a transplanted conduit) in group 1.

FIG. 14C shows a regeneration condition of a nerve fiber after immunohistochemical staining with a nerve fiber marker protein NF-200 on a longitudinal tissue slice of a sciatic nerve injury portion (including a transplanted conduit) in group 5.

FIG. 14D shows a regeneration condition of a nerve fiber after immunohistochemical staining with a nerve fiber marker protein NF-200 on a longitudinal tissue slice of a sciatic nerve injury portion (including a transplanted conduit) in group 4.

FIG. 14E shows a regeneration condition of a nerve fiber after immunohistochemical staining with a nerve fiber marker protein NF-200 on a longitudinal tissue slice of a sciatic nerve injury portion (including a transplanted conduit) in group 3.

FIG. 14F shows a regeneration condition of a nerve fiber after immunohistochemical staining with a nerve fiber marker protein NF-200 on a longitudinal tissue slice of a sciatic nerve injury portion (including a transplanted conduit) in group 2.

FIG. 15A shows a density of regenerated nerve fibers under immunohistochemical staining with a nerve fiber marker protein NF-200 on a midpoint cross-sectional tissue slice of a sciatic nerve of a lower limb in a normal group.

FIG. 15B shows a density of regenerated nerve fibers under immunohistochemical staining with a nerve fiber marker protein NF-200 on a midpoint cross-sectional tissue slice of a sciatic nerve injury portion (conduit transplantation portion) in group 1.

FIG. 15C shows a density of regenerated nerve fibers under immunohistochemical staining with a nerve fiber marker protein NF-200 on a midpoint cross-sectional tissue slice of a sciatic nerve injury portion (conduit transplantation portion) in group 5.

FIG. 15D shows a density of regenerated nerve fibers under immunohistochemical staining with a nerve fiber marker protein NF-200 on a midpoint cross-sectional tissue slice of a sciatic nerve injury portion (conduit transplantation portion) in group 4.

FIG. 15E shows a density of regenerated nerve fibers under immunohistochemical staining with a nerve fiber marker protein NF-200 on a midpoint cross-sectional tissue slice of a sciatic nerve injury portion (conduit transplantation portion) in group 3.

FIG. 15F shows a density of regenerated nerve fibers under immunohistochemical staining with a nerve fiber marker protein NF-200 on a midpoint cross-sectional tissue slice of a sciatic nerve injury portion (conduit transplantation portion) in group 2.

### DETAILED DESCRIPTION

### EXAMPLE 1

### Optimization Selection of Stripes

1. Making of Micropatterns on Material Surfaces

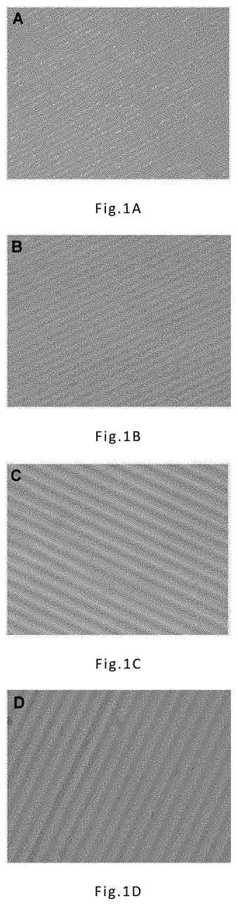

Electron beam lithography and nanoimprint lithography technologies were adopted. Firstly, the surface of a 3×3 cm silicon wafer was spin-coated with a polymethyl methacrylate (PMMA) film, and stripe type micropatterns with the same width, spacing and height were etched on the surface of the PMMA film by the electron beam lithography technology. With a micropatterned substrate as a template, a polydimethylsiloxane (PDMS) base material and a curing agent were mixed in the ratio of 10:1 and dropwise added to the surface of the template (0.5 mL/cm²), the template was dried at 60° C. for 4 h in a vacuum drying oven, the PDMS was solidified on the surface of the template to form a film, and the PDMS film was peeled off from the template, so that patterns (see FIG. 1A˜FIG. 1D) complementary to the micropatterns of the template were formed on the surface of the PDMS film. The experimental stripes were equal in width, height and spacing and had the specifications including: 0.5 μm×0.5 μm×0.5 μm (0.5 μm), 1.0 μm×1.0 μm×1.0 μm (1.0 μm), 1.5 μm×1.5 μm×1.5 μm (1.5 μm), 2.0 μm×2.0 μm×2.0 μm (2.0 μm), 2.5 μm×2.5 μm×2.5 μm (2.5 μm), and 3.0 μm×3.0 μm×3.0 μm (3.0 μm).

2. Material taking, Culture, Amplification and Authentication of EMSCs

An SD rat (80-100 g) was anesthetized by intraperitoneal injection of 10% chloral hydrate (330 g/kg), the whole body skin was disinfected, the skin and a nasal bone were cut through nostrils up to an inner canthus along a nasal cavity under sterile conditions to expose nasal septum mucosa, ⅓ of the nasal septum was cut off and placed in a PBS, and full-thickness nasal mucosa was peeled off. The nasal mucosa of the SD rat was taken out and then rinsed with a serum-free DMEM/F12 mixed medium (containing 200 U/mL penicillin and 200 U/mL streptomycin) for three times to remove blood stains. The nasal mucosa was placed in a DMEM/F12 medium containing 10% (m/m) fetal bovine serum (i.e., a common complete medium containing 100 U/mL penicillin and 100 U/mL streptomycin) and fully cut up with eye scissors. The cut-up nasal mucosa was digested by 0.25% trypsin at 37° C. for 15 min. After centrifugation was performed and a supernatant was discarded, cells and small tissue blocks were inoculated in a Corning culture flask and cultured in a CO₂ incubator (37° C., 5% CO₂ and saturated humidity). A new DMEM/F12 medium containing 10% (m/m) fetal bovine serum was supplemented after 3 days of cell culture. After that, half of the solution was changed once every three days, and the cells were digested and passed when the bottom of the flask was covered.

A 24-well culture plate was inoculated with the fifth generation cells. Immunofluorescent staining was performed respectively with marker proteins vimentin, Nestin, CD133, CD44 and antibodies of EMSCs, and the cultured cells were authenticated as the EMSCs. Operation steps were as follows: after being fixed by a paraformaldehyde solution with the concentration of 4%, the cells were closed at 37° C. in a 0.25% TritonX-100 and 3% bovine serum albumin (BSA) mixed solution for 30 min, incubation was performed with a first antibody at 4° C. for 12 h, incubation was performed with a Cy3-marked corresponding second antibody at room temperature for 1 h after rinsing with a PBS, PBS rinsing was performed for 3 times, cell nuclei were counter-stained with Hoc hest33342, rinsing was performed with the PBS, a slide was sealed with neutral glycerine, the slide was observed under a Leica fluorescence microscope and shot, and for negative control, the first antibody was replaced with a PBS, while other steps were as above. Other cells were used for following experiments.

3. Optimization Selection of Stripes on Film Surfaces

The induction differentiation effect from stripe-induced EMSCs to Schwann cell-like cells was used as a standard for optimization selection of the stripes. The fifth generation EMSCs cultured and authenticated above were digested with trypsin, the cells were collected, and the cell density was adjusted to be about 1×10⁵ cells/mL. The cells were planted on the surface of the micropatterned PDMS film (surrounded by paraffin ridges to limit the medium and cell loss) flatly-laid in the culture plate with the planting amount of 0.5 mL/cm². The EMSCs/micropatterned PDMS film was placed in a CO₂ incubator (37° C., 5% CO₂ and saturated humidity) and cultured with DMEM/F12 (containing 100 U/mL penicillin and 100 U/mL streptomycin) containing 10% fetal bovine serum. The EMSCs were attached to the surface of the micropatterned PDMS film after 2 h. Then a new DMEM/F12 medium containing 10% fetal bovine serum was fully added to the culture plate to continue culture, and half of the solution was changed once every three days. After 14 days, a cell/micropattern film compound was fixed with a 4% paraformaldehyde solution, immunofluorescent staining was performed with antibodies of Schwann cell marker proteins S100 and MBP, and the differentiation of EMSCs into Schwann cells on the micropattern film was observed. Western blotting was used to detect the relative content of the Schwann cell marker proteins, the effects of several stripes on differentiation of the EMSCs into the Schwann cells were compared, and the stripes with the strongest inducing ability were selected as the patterns for modifying biomaterial scaffolds. In the above experiment process, the EMSCs were cultured only in the DMEM/F12 medium containing 10% fetal bovine serum without any inducer to obtain the induction differentiation effect of the single factor of the stripes. The biomaterial film, with or without planted EMSCs, with the surface provided with the stripes of this specification was used as a scaffold material for making a nerve conduit.

4. Result Analysis

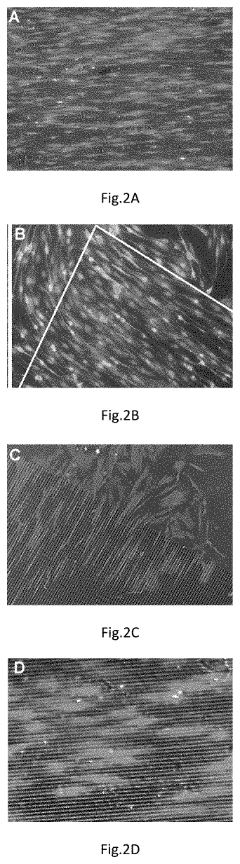

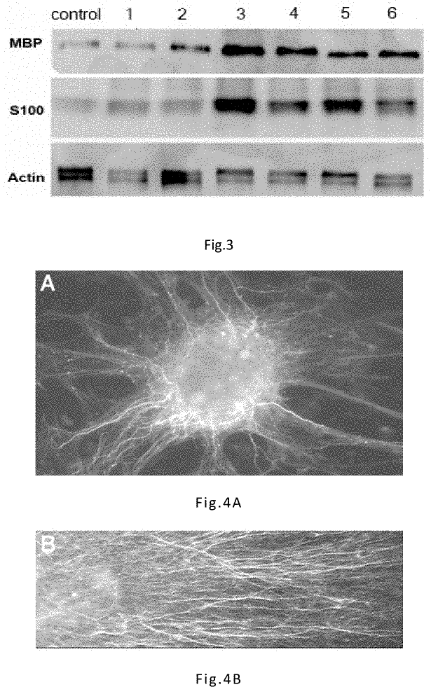

Immunofluorescent staining results of the Schwann cell marker proteins S100 and MBP show that the EMSCs are planted on the surfaces of the PDMS films with the stripes of various specifications and cultured in the DMEM/F12 medium containing 10% fetal bovine serum, the cells are like Schwann cells in morphology and are arranged in parallel along the stripes. The morphology and staining intensity of the cells on the film surfaces of the stripes of different specifications are different (see FIG. 2A˜FIG. 2D). The relative content of the Schwann cell marker proteins is detected by the Western blotting method, the effects of several stripes on differentiation of the EMSCs into the Schwann cells are compared, and the results show that the 1.0 μm×1.0 μm×1.0 μm stripes have the strongest ability to induce the EMSCs to differentiate into the Schwann cells (see Table 1 and FIG. 3). The PDMS films without stripes have the weakest inducing ability. Therefore, in following experiments, the 1.0 μm×1.0 μm×1.0 μm stripe films are adopted to be used as a cell growth substrate and a nerve conduit material. The relative content of the Schwann cell marker proteins MBP and S100 in a 1 μm stripe set is obviously higher than that of other sets (p<0.05, and n=3).

TABLE 1
Comparison of relative content (ratios to Actin) of Schwann cell marker proteins
expressed by stripe-induced EMSCs ( X̅ ± SD)

| Schwann cell | Blank | Non-stripe | 0.5 μm | 1 μm | 1.5 μm | 2 μm | 2.5 μm |
|---|---|---|---|---|---|---|---|
| marker proteins | Control | films | stripes | stripes | stripes | stripes | stripes |
| MBP | 0.15 ± 0.05 | 0.33 ± 0.11 | 0.41 ± 0.07 | 1.57 ± 0.12 | 1.12 ± 0.07 | 0.77 ± 0.13 | 0.79 ± 0.11 |
| S100 | 0.17 ± 0.09 | 0.91 ± 0.13 | 0.87 ± 0.15 | 1.99 ± 0.35 | 1.17 ± 0.13 | 1.21 ± 0.27 | 0.87 ± 0.33 |

### EXAMPLE 2

### Practical Application of Micropatterned PCL Film

1. Preparation of PCL Film with Micropatterned Surface

Electron beam lithography and nanoimprint lithography technologies were adopted. Firstly, the surface of a 3×3 cm silicon wafer was spin-coated with a polymethyl methacrylate (PMMA) film, and stripe type micropatterns with the width, spacing and height all being 1 μm were etched on the surface of the PMMA film by the electron beam lithography technology. With a micropatterned substrate as a template, a PDMS base material and a curing agent were mixed in the ratio of 10:1 and dropwise added to the surface of the template, the template was dried at 60° C. for 4 h in a vacuum drying oven, the PDMS was solidified on the surface of the template to form a film, and the PDMS film was peeled off from the template, so that patterns complementary to the micropatterns of the template were formed on the surface of the PDMS film. With the above micropatterned PDMS film as a template, a polycaprolactone (PCL)-dichloromethane solution with the volume concentration of 20% was mixed and dropwise added to the surface of the PDMS template (0.5 mL/cm²), the template was dried in the vacuum drying oven for 1 h, PCL was solidified on the surface of the PDMS template to form a film, and the PCL film was peeled off from the template, so that patterns complementary to the micropatterns of the template were formed on the surface of the PCL film.

2. Material taking, Culture, Amplification and Authentication of EMSCs

An SD rat (80-100 g) was anesthetized by intraperitoneal injection of 10% chloral hydrate according to the dosage of 330 g/kg, the whole body skin was disinfected, the skin and a nasal bone were cut through nostrils up to an inner canthus along a nasal cavity under sterile conditions to expose nasal septum mucosa, ⅓ of the nasal septum was cut off and placed in a PBS, and full-thickness nasal mucosa was peeled off. The nasal mucosa of the SD rat was taken out and then rinsed with a serum-free DMEM/F12 mixed medium (containing 200 U/mL penicillin and 200 U/mL streptomycin) for three times to remove blood stains. The nasal mucosa was placed in a DMEM/F12 medium containing 10% fetal bovine serum (i.e., a common complete medium containing 100 U/mL penicillin and 100 U/mL streptomycin) and fully cut up with eye scissors. The cut-up nasal mucosa is digested by trypsin with the mass concentration of 0.25% at 37° C. for 15 min. After centrifugation was performed and a supernatant was discarded, cells and small tissue blocks were inoculated in a Corning culture flask and cultured in a CO₂ incubator (37° C., 5% CO₂ and saturated humidity). A new DMEM/F12 medium containing 10% fetal bovine serum was supplemented after 3 days of cell culture. After that, half of the solution was changed once every three days, and the cells were digested and passed when the bottom of the flask was covered.

A 24-well culture plate was inoculated with the fifth generation cells. Immunofluorescent staining was performed respectively with marker proteins vimentin, Nestin, CD133, CD44 and antibodies of EMSCs, and the cultured cells were authenticated as the EMSCs. Operation steps were as follows: after being fixed by a 4% paraformaldehyde solution, the cells were closed at 37° C. in a 0.25% TritonX-100 and 3% bovine serum albumin (BSA) mixed solution for 30 min, incubation was performed with a first antibody at 4° C. for 12 h, incubation was performed with a Cy3-marked corresponding second antibody at room temperature for 1 h after rinsing with a PBS, rinsing is performed with the PBS for 3 times, cell nuclei were counter-stained with Hoc hest33342, rinsing was performed with the PBS, then a slide was sealed with neutral glycerine, and the slide was observed under a Leica fluorescence microscope and shot. For negative control, the first antibody was replaced with a PBS, while other steps were as above. Other cells were used for the following steps.

3. Planting of EMSCs on Surface of Micropatterned PCL Film

The above EMSCs were digested with trypsin, the cells were collected, and the cell density was adjusted to be about 1×10⁵ cells/mL. The cells were planted on the micropatterned PCL film (surrounded by paraffin ridges to limit the medium and cell loss) with the density of 0.5 mL/cm². The PCL film was placed in a CO₂ incubator (37° C., 5% CO₂ and saturated humidity) and cultured with a DMEM/F12 medium containing 10% fetal bovine serum, and a culture solution is changed once every three days. After 14 days of culture, an EMSCs/micropattern film compound was fixed with a 4% paraformaldehyde solution, immunofluorescent staining was performed with antibodies of Schwann cell marker proteins S100 and MBP, and the growth condition of Schwann cells differentiated from the EMSCs on the micropattern film was observed.

4. Culture of Rat Embryo Neural Stem Cells

The SD rat with 14-16 days of gestation was anesthetized and then an embryo was taken out. Cerebral cortex tissue with the size of about 0.5 mm×1 mm×2 mm on the two sides was taken. Pia mater was removed completely, and the tissue was washed twice in a serum-free DMEM/F12 mixed medium (containing 200 U/mL penicillin and 200 U/mL streptomycin). The taken tissue was washed in a PBS, cut up, digested with trypsin and filtered by a screen to prepare a single-cell suspension. A neural stem cell medium (2% B27, 20 ng/mL bFGF, 20 ng/mL EGF, penicillin and streptomycin each being 100 U/mL were added into the DMEM/F12 medium) was inoculated with the single-cell suspension with the inoculation density of 2×10⁵ cells/mL. In order to make sure proliferation of neural stem cell spheres, the obtained inoculation density of the stem cell spheres was 2,000 spheres/mL. Afterwards, passage was performed once every 1-2 weeks via a mechanical digestion method, and multiple times of passage were performed. Neural spheres and differentiated cells were fixed for 30 min at room temperature with a 4% paraformaldehyde solution, and immunofluorescent staining authentication was performed by using an antibody of a neural stem cell marker protein Nestin. The remaining neural stem cells were used in the following experiments to simulate the process of promoting nerve regeneration in vivo: (1) neural spheres or scattered neural stem cells were planted in the surface of a striped (1.0 μm×1.0 μm×1.0 μm) PCL film, and after 21 days of culture with a neural stem cell medium, the growth condition of nerve fibers along stripes was observed by immunofluorescent staining with an antibody of a nerve fiber marker protein NF-200; the neural stem cells were differentiated into nerve cells on the surface of a non-striped PCL film and grew radially; the nerve fibers grew in parallel on the surface of the striped (1.0 μm×1.0 μm×1.0 μm) PCL film; and in order to show the stripes and the nerve fibers at the same time, the scattered neural stem cells were planted on the surface of the striped (1.0 μm×1.0 μm×1.0 μm) PCL film, and it can be seen that the nerve fibers grew along the stripes (see FIG. 4A˜FIG. 4C); and (2) the neural stem cells were planted on the EMSCs/PCL micropattern film, and the growth condition of the nerve fibers on the surface of the cell/striped film was observed.

5. Planting of Neural Stem Cells on EMSCs/PCL Micropattern Film

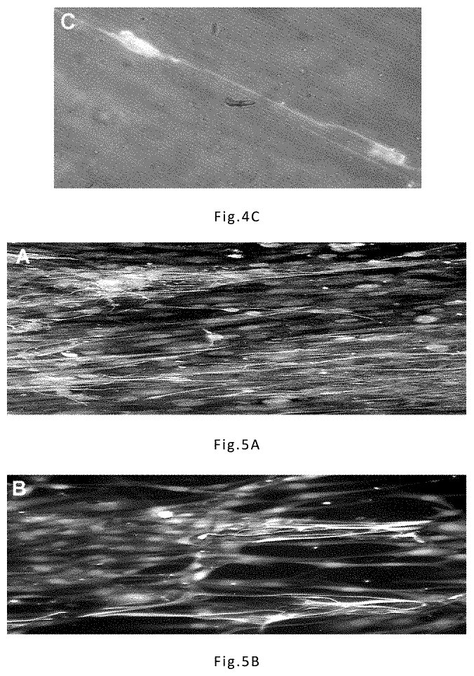

The above cultured secondary neural spheres were partially scattered and planted on the surface of the EMSCs/PCL composite film, and then the film was cultured in a CO₂ incubator (37° C., 5% CO₂ and saturated humidity) with the neural stem cell medium. After 14 days of culture, a neural stem cell/EMSCs/micropattern film compound was fixed with a 4% paraformaldehyde solution, immunofluorescent double-marked staining was performed with an antibody of neural cell/Schwann cell marker proteins NF-200/MBP, and differentiation of the neural stem cells on the EMSCs/micropattern film and the parallel growth condition of the nerve fibers were observed. Nerve cells grew in parallel on the surface of the EMSCs (Schwann cells)/striped micropattern PCL film (Schwann cells at the bottom layer were stained with immunofluorescence of the marker protein S100 (fluorescein 488, green); and the nerve fibers at the upper layer were stained with immunofluorescence of the marker protein NF-200 (cy3, red)) (FIG. 5A and FIG. 5B).

6. In Vivo Transplantation Experiment

(1) Experimental Animals and Transplantation Operation Process

50 healthy adult male SD rats with the weight of 250-300 g were randomly divided into 5 groups with 10 rats in each group. Group 1 was a simple sciatic nerve injury group; group 2 was a sciatic nerve injury+transplanted simple non-stripe PCL conduit group; group 3 was a sciatic nerve injury+transplanted EMSCs/non-stripe PCL conduit group; group 4 was a sciatic nerve injury+transplanted simple striped PCL conduit group; and group 5 was a sciatic nerve injury+transplanted EMSCs/striped PCL conduit group.

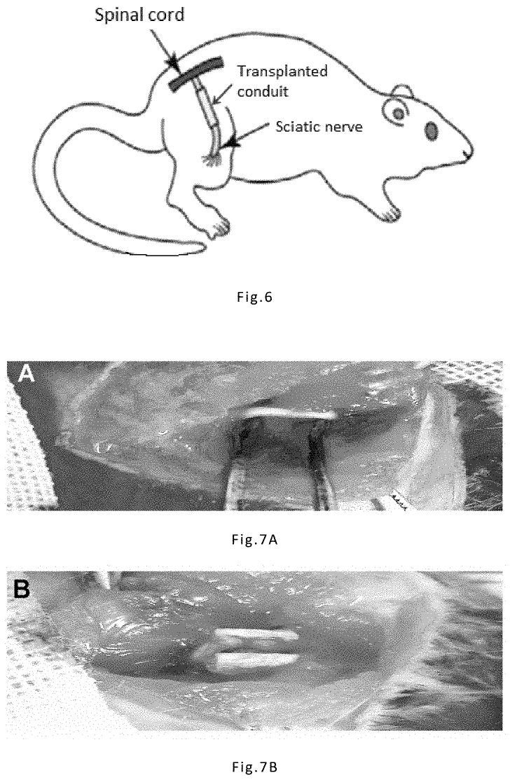

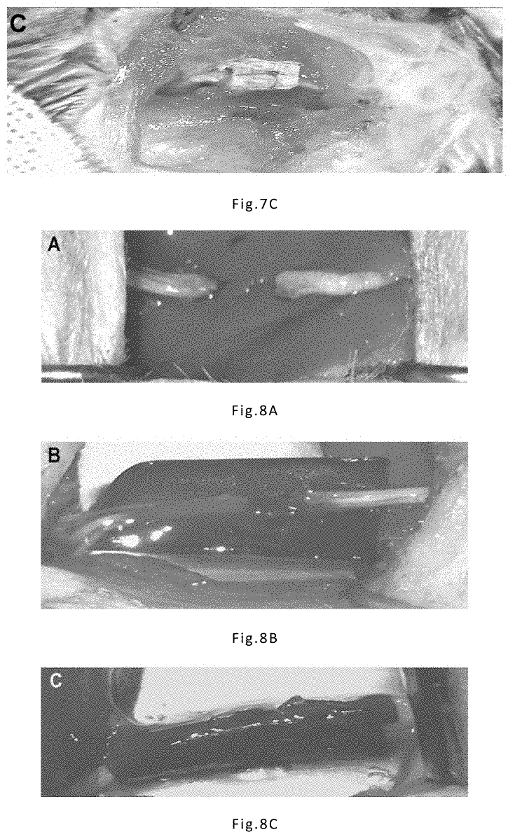

The operation process on the animals was as follows: 10% chloral hydrate was adopted for intraperitoneal anesthesia at 400 mg/kg, and the center of posterior femur was cut open to expose the sciatic nerve in the middle section of the right hind limb. In group 1, the sciatic nerves were excised by 6 mm and then muscles and skins were sutured directly. In group 2, the simple non-stripe PCL conduits were transplanted in sciatic nerve defect portions. In group 3, the EMSCs (Schwann cells)/non-stripe PCL conduits were transplanted in sciatic nerve defect portions. In group 4, the simple striped (1.0 μm×1.0 μm×1.0 μm) PCL conduits were transplanted in sciatic nerve defect portions. In group 5, EMSCs (Schwann cells)/striped (1.0 μm×1.0 μm×1.0 μm) PCL conduits were transplanted in sciatic nerve defect portions. After conduit transplantation, fibrin glue was used to seal anastomotic stomas and muscles and skins were sutured (see FIGS. 6, 7A˜7C and 8A˜8C). All groups were conventionally bred after operations, and sciatic nerve indexes were regularly measured.

(2) Evaluation Indexes for Effects of Repairing Nerve Injury by Conduit Transplantation

1) Observation of General Conditions and Measurement of Sciatic Functional Index (SFI)

The diet, foot ulcer, limb activity and incision healing conditions of the rats were observed conventionally after the operations. The SFI was measured each week: a two-end-open wood trough which is 60 cm long, 10 cm wide and 20 cm high was manufactured, and 70 g/m² white paper was cut to be equal to the wood trough in length and width and then laid at the bottom of the trough. Bilateral hind limbs of each rat were dipped in pigment to color double ankle joints, the rat was placed at one end of the trough and made to walk to the other end of the trough by itself, and 5-6 footprints were left by the hind limb on each side. Three indexes of normal feet (N) and injured feet (E) were respectively measured in selected clear footprints: A, PL (print length), B, TS (toe spread) and C, IT (intermediary toe spread). The above indexes were substituted into a Bain formula, and the SFI was worked out.

SFI=109.5(ETS−NTS)/NTS−38.3(EPL−NPL)/NPL+13.3(EIT−NIT)/NIT−8.8. Bain formula:

SFI=0 means normal, and −100 means complete injury. Measurement results of the SFI of the sciatic nerve injury sides of each group 16 weeks after the animal operations are shown in Table 2. The SFI of the injury sides in group 5 is obviously higher than that in other groups (p<0.05 and n=9).

TABLE 2
Comparison of SFI of sciatic nerve injury sides in each group ( X̅ ± SD)

| Group 1 | Group 2 | Group 3 | Group 4 | Group 5 |
|---|---|---|---|---|
| −91 ± 25 | −77 ± 31 | −68 ± 19 | −57 ± 23 | −37 ± 17 |

2) Fluorochrome Retrograde Tracing

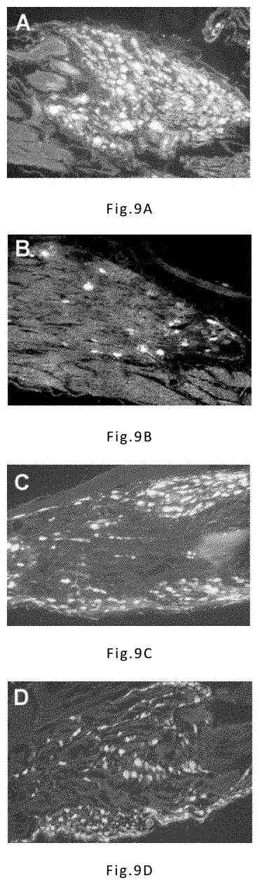

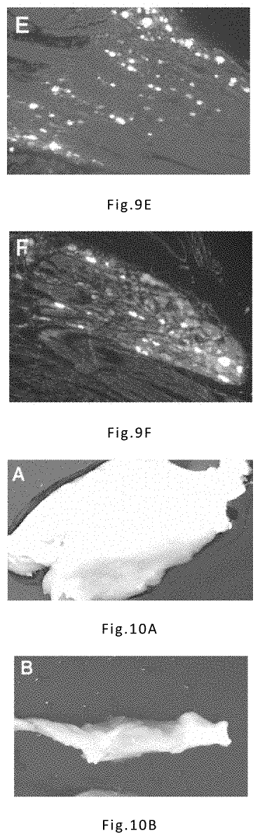

Three rats were randomly selected from each group for fluorochrome retrograde tracing 15 weeks after the operations (1 week before the observation end point). The sciatic nerves were exposed again after anesthesia, and 2 μL of a 5% fluorochrome-phosphate buffer saline (PBS) solution was injected with a microsyringe at the 5 mm position of the far end of a transplant. The same amount of fluorochrome was also injected to the corresponding positions of the sciatic nerves on the normal sides. Operative incisions were sutured, and the animals continued to be bred. Corresponding L4-L6 and S1-S2 dorsal root ganglions on the left and right sides were taken out 1 week later and longitudinally sliced with the thickness of 10 μm by a freezing microtome. Ten consecutive slices were observed respectively under a fluorescence microscope (since ganglions are small, the complete picture can be observed within a lower power field), the total number of positive cells labeled with fluorochrome in each slice was counted by using Image-proPlus6.0, and an average value was calculated. The positive cell ratio was labeled on double sides (positive cell ratio=positive cell number labeled on the experimental side/positive cell number on the control side×100%) and configured to reflect the nerve regeneration degree (the total number of the positive cells is positively correlated to the conduit transplantation repairing effect, and results are shown in Table 3 and FIG. 9A˜FIG. 9F). The ratio of the number of the ganglion positive cells on the injury sides to that of the ganglion positive cells on the normal sides in group 5 is obviously higher than that in other groups (p<0.05 and n=9).

TABLE 3
Ratio of number of fluorochrome-labeled positive cells of dorsal root
ganglions on sciatic nerve injury sides to number of cells on normal
sides in each group ( X̅ ± SD)

| Group 1 | Group 2 | Group 3 | Group 4 | Group 5 |
|---|---|---|---|---|
| 0.07 ± 0.02 | 0.13 ± 0.07 | 0.21 ± 0.15 | 0.37 ± 0.11 | 0.57 ± 0.12 |

3) Wet Weight Measurement and Morphological Observation of Gastrocnemii

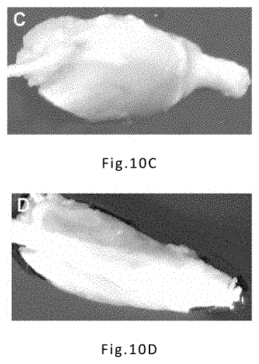

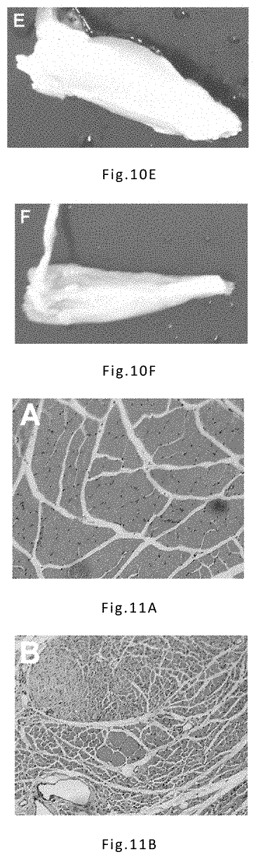

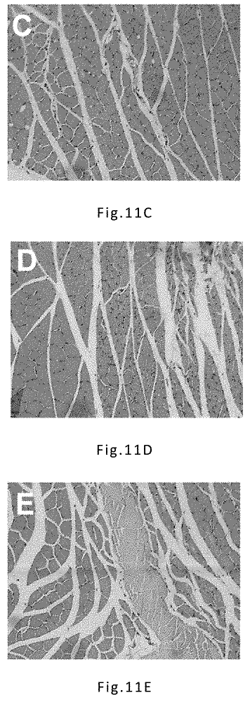

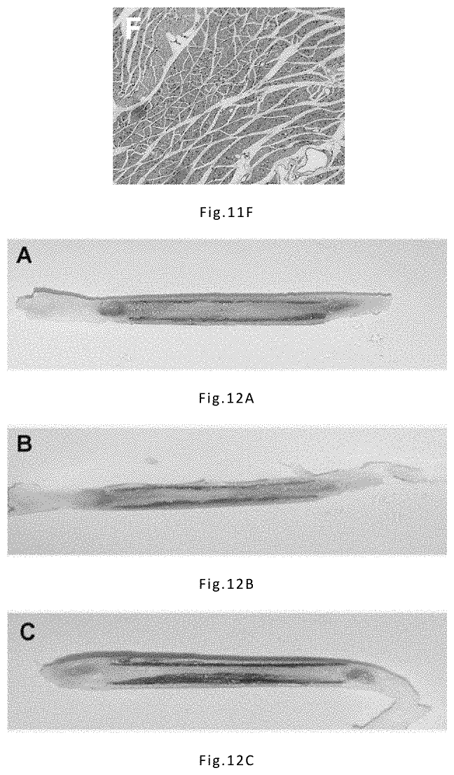

16 weeks after the animal operations, the animals were anesthetized, bilateral gastrocnemii were completely cut and weighed with an electronic balance (accurate to 0.001 g), and the wet weight ratio of the bilateral gastrocnemii of the animals in each group was calculated (wet weight ratio=wet weight of experimental side muscle/wet weight of control side muscle×100%). The results are shown in Table 4. After weighing, the muscle was fixed with a 4% paraformaldehyde solution and embedded in conventional paraffin. The tissue slices were stained with H-E and observed under a light microscope. The cross-sectional area of left and right gastrocnemius fibers was measured by a Leica microscopic image analysis system, and the cross-sectional area ratio (cross-sectional area ratio=cross-sectional area of experimental side muscle/cross-sectional area of control side muscle×100%) was calculated. The results are shown in Table 5, FIG. 10A˜FIG. 10F and FIG. 11A˜FIG. 11F. The wet weight ratio of the gastrocnemii in group 5 is obviously higher than that in other groups (p<0.05 and n=9), and the fiber cross-sectional area ratio of the gastrocnemii in group 5 is obviously higher than that of the gastrocnemii in other groups (p<0.05 and n=9).

TABLE 4
Comparison of ratio of wet weight of gastrocnemii on sciatic nerve injury
sides to that of gastrocnemii on normal sides in each group ( X̅ ± SD)

| Group 1 | Group 2 | Group 3 | Group 4 | Group 5 |
|---|---|---|---|---|
| 0.19 ± 0.07 | 0.31 ± 0.13 | 0.41 ± 0.13 | 0.63 ± 0.09 | 0.77 ± 0.18 |

TABLE 5
Ratio of fiber cross-sectional area of gastrocnemii on sciatic nerve injury
sides to that of gastrocnemii on normal sides in each group ( X̅ ± SD)

| Group 1 | Group 2 | Group 3 | Group 4 | Group 5 |
|---|---|---|---|---|
| 0.21 ± 0.09 | 0.29 ± 0.13 | 0.47 ± 0.17 | 0.75 ± 0.12 | 0.87 ± 0.23 |

4) Morphological Observation and Measurement of Sciatic Nerves

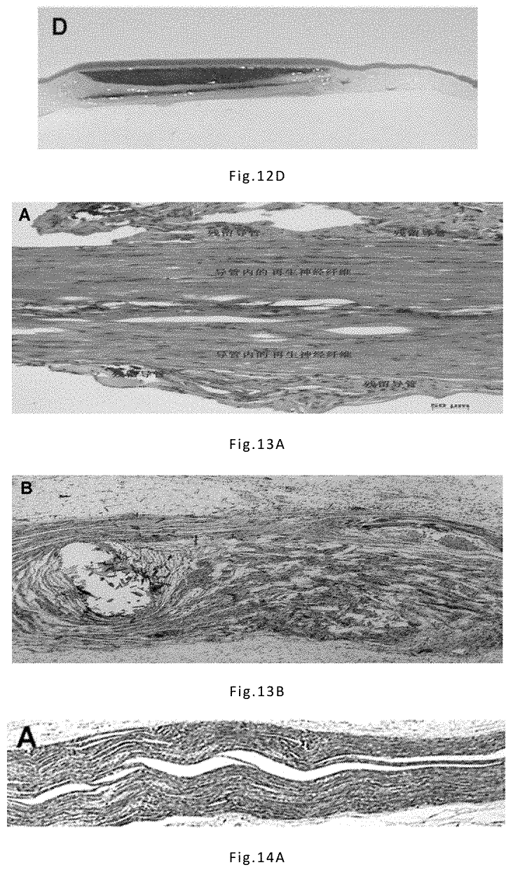

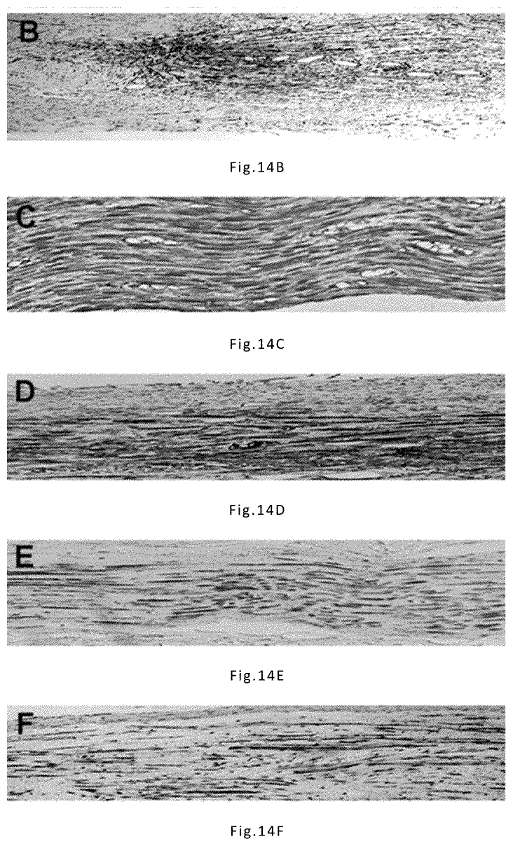

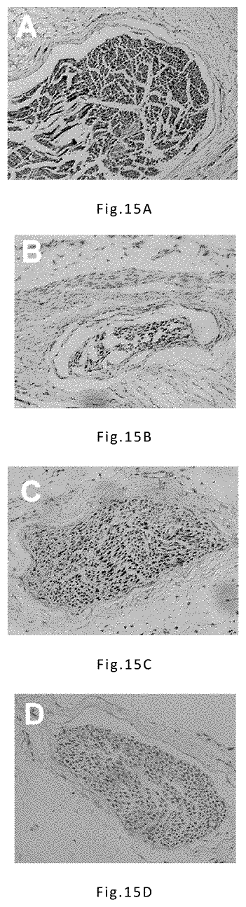

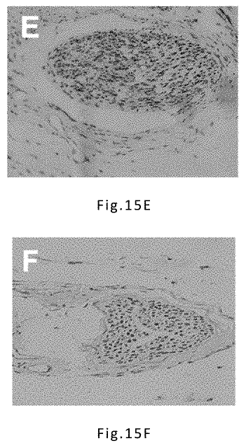

16 weeks after the animal operations, after the animals were anesthetized, the original incisions were cut open to expose the sciatic nerves, and the regeneration condition of the sciatic nerves was observed (FIGS. 12A˜12D). The rats with and without the transplanted nerve conduits after sciatic nerve injury were selected, each sciatic nerve after injury repairing included a near section (upper section), an injured section (conduit transplantation portion) and a far section (lower section) of the injured portion, and after being fixed with a 4% paraformaldehyde solution, the nerves were embedded in conventional paraffin and sliced. The slicing direction was parallel to a nerve longitudinal axis in the longitudinal direction and passed the near section (upper section), the injured section (conduit transplantation portion) and the far section (lower section), so that the condition of the regenerated nerve fibers passing the conduits was conveniently observed. Tissue slices were stained with H.E (FIGS. 13A˜13B). In group 5, the sciatic nerves at the injury near ends had grown into the nerve conduit and reached the far side through the conduits after treatment, and no residual cavity was found after absorbable sutures were absorbed. In the untreated group, severe degeneration of the nerve fibers was seen, and only a small number of regenerated nerve fibers were seen (the upper side of the figure), and cavities were residual after absorbable sutures were absorbed. Immunohistochemical staining was performed with an antibody of the nerve fiber marker protein NF-200 (FIGS. 14A˜14F). Midpoint cross-section tissue at normal sciatic nerves and the injured portions (conduit transplantation portions) of the sciatic nerves in other groups was sliced, and the density of the regenerated nerve fibers observed by immunohistochemical staining of the nerve fiber marker protein NF-200 was shown in FIGS. 15A˜15F. After microscopic observation and image collection, the density of the nerve fibers was measured by an image analysis system (the thickest sections of the longitudinal sections of the sciatic nerves in each group of animals were selected for comparison). The results are shown in Table 6. The ratio of the number of the nerve fibers on the injury sides to that of the nerve fibers on the normal sides in group 5 is obviously higher than that in other groups (p<0.05 and n=9).

TABLE 6
Ratio of number of regenerated nerve fiber cross-sections on sciatic
nerve injury sides to that of cross-sections on normal sides in each
group ( X̅ ± SD)

| Group 1 | Group 2 | Group 3 | Group 4 | Group 5 |
|---|---|---|---|---|
| 0.18 ± 0.08 | 0.21 ± 0.07 | 0.35 ± 0.17 | 0.57 ± 0.21 | 0.69 ± 0.19 |

### EXAMPLE 3

### Practical Application of Micropatterned Fibrous Protein/Chitosan Composite Film

1. Preparation of Fibrous Protein/Chitosan Composite Film with Micropatterned Surface

Fibrinogen and chitosan have good biocompatibility, can be mixed to enhance the mechanical strength of the composite film, and can be crosslinked with one or more cell growth factors such as EGF, FGE, NGF and SHH through biological crosslinking agents such as genipin or/and glutamine transaminase (TG) to construct drug sustained release scaffolds so as to further improve their function of promoting nerve regeneration. Firstly, the fibrinogen/chitosan composite film was selected as the material to make the striped nerve conduits, and after planting of the EMSCs or not, the film was used to be transplanted in vivo to repair sciatic nerve injury, and the application effect was evaluated. The nerve conduit material without planting of the EMSCs was used as control. A making process of a striped fibrinogen solution/chitosan composite film is as follows.

A fibrinogen aqueous solution with the concentration of 5% and a chitosan acetate solution with the concentration of 2% were prepared, and then the fibrinogen solution and the chitosan solution were mixed evenly according to the mass ratio of 9:1. The prepared solution was dropwise added to a PDMS film (0.5 mL/cm², and surrounded by paraffin ridges to limit fluid loss) with the surface modified with 1.0 μm parallel stripes and pre-laid in a culture plate, after the liquid was leveled, 50 μL (5 U) of thrombin (100 U/mL) was added through a micro sprayer, and 50 μL of 1% genipin was added 5 minutes later. The culture plate was placed in a drying oven and cured at 37° C., and the liquid was solidified into gel after 12 h. At the moment, a 50 g weight pressed the gel, the gel continued to be vacuum-dried at 25° C. until no flowing liquid existed on the film surface, but the film surface was kept moist. Then the film and a template were placed in a refrigerator and cured at 4° C. to be stabilized for 24 h. The cured fibrous protein/chitosan composite film was slowly and carefully peeled off from the template to ensure the integrity of the film and stripes. At this time, patterns complementary to template micropatterns were formed on the surface of the fibrous protein/chitosan composite film.

2. Planting of EMSCs on Surface of Micropatterned Fibrous Protein/Chitosan Composite Film

In order to verify that stripes on the surfaces of films of other materials also had the effect of inducing the EMSCs to differentiate to Schwann cell-like cells, the above EMSCs were digested with trypsin, the cells were collected, and the cell density was adjusted to be about 1×10⁵ cells/mL. The cells were planted on the surface of the micropatterned fibrous protein/chitosan composite film (surrounded by ridges to limit the medium and cell loss) with the density of 0.5 mL/cm². The film was placed in a CO₂ incubator (37° C., 5% CO₂ and saturated humidity) and cultured with a common medium. After 14 days of culture, an EMSCs/micropattern fibrous protein/chitosan film compound was fixed with a 4% paraformaldehyde solution, immunofluorescent staining was performed with antibodies of Schwann cell marker proteins S100 and MBP, and the growth condition of Schwann cells differentiated from the EMSCs on the micropattern film was observed.

3. Culture of Rat Embryo Neural Stem Cells

The SD rat with 14-16 days of gestation was anesthetized and then an embryo was taken out. Cerebral cortex tissue with the size of about 0.5 mm×1 mm×2 mm on the two sides was taken. Pia mater was removed completely, and the tissue was washed twice in a serum-free DMEM/F12 mixed medium (containing 200 U/mL penicillin and 200 U/mL streptomycin). The taken tissue was washed in a PBS, cut up, digested with trypsin and filtered by a screen to prepare a single-cell suspension. A neural stem cell medium (a 2% cell culture additive B27, 20 ng/mL bFGF, 20 ng/mL EGF, penicillin and streptomycin each being 100 U/mL were added into the DMEM/F12 medium) was inoculated with the single-cell suspension with the inoculation density of 2×10⁵ cells/mL. In order to make sure proliferation of neural stem cell spheres, the obtained inoculation density of the stem cell spheres was 2,000 spheres/mL. Afterwards, passage was performed once every 1-2 weeks via a mechanical digestion method, and multiple times of passage were performed. Neural spheres and differentiated cells were fixed for 30 min at room temperature with a 4% paraformaldehyde solution, and immunofluorescent staining authentication was performed by using an antibody of a neural stem cell marker protein Nestin. The remaining neural stem cells were used in the following experiments to simulate the process of promoting nerve regeneration in vivo: (1) neural spheres or scattered neural stem cells were planted in the surface of a striped (1.0 μm×1.0 μm×1.0 μm) fibrous protein/chitosan micropattern film and cultured with the neural stem cell medium, and after 21 days, the growth condition of nerve fibers along stripes was observed by immunofluorescent staining with an antibody of a nerve fiber marker protein NF-200; and (2) the neural stem cells were planted on the EMSCs/fibrous protein/chitosan micropattern film, and the growth condition of the nerve fibers on the surface of the cell/striped film was observed (described below).

4. Planting of Neural Stem Cells on EMSCs/Fibrous Protein/Chitosan Micropattern Film

The cultured secondary neural spheres were partially scattered and planted on the surface of the EMSCs/PCL micropattern film, and then the film was cultured in the CO₂ incubator (37° C., 5% CO₂ and saturated humidity) with DMEM/F12 (containing 100 U/mL penicillin and 100 U/mL streptomycin) containing 10% fetal bovine serum. After 14 days of culture, a neural stem cell/EMSCs/fibrous protein/chitosan micropattern film compound was fixed with a 4% paraformaldehyde solution, immunofluorescent double-marked staining was performed with antibodies of neural cell/Schwann cell marker proteins NF-200/MBP, and differentiation of the neural stem cells on the EMSCs/fibrous protein/chitosan micropattern film and the parallel growth condition of the nerve fibers were observed.

5. In Vivo Transplantation Experiment

(1) Experimental Animals and Transplantation Operation Process

50 healthy adult male SD rats with the weight of 250-300 g were randomly divided into 5 groups with 10 rats in each group. Group 1 was a simple sciatic nerve injury group; group 2 was a sciatic nerve injury+transplanted simple non-stripe fibrous protein/chitosan micropattern conduit group; group 3 was a sciatic nerve injury+transplanted EMSCs/non-stripe fibrous protein/chitosan micropattern conduit group; group 4 was a sciatic nerve injury+transplanted simple striped fibrous protein/chitosan micropattern conduit group; and group 5 was a sciatic nerve injury+transplanted EMSCs/striped fibrous protein/chitosan micropattern conduit group.

The operation process on the animals was as follows: 10% chloral hydrate was adopted for intraperitoneal anesthesia at 400 mg/kg, and the center of posterior femur was cut open to expose the sciatic nerve in the middle section of the right hind limb. The five experiment groups were set: in group 1, the sciatic nerves were excised by 6 mm and then muscles and skins were sutured directly; in group 2, the simple non-stripe fibrous protein/chitosan micropattern conduits were transplanted in sciatic nerve defect portions; in group 3, the EMSCs/fibrous protein/chitosan micropattern non-stripe conduits were transplanted in sciatic nerve defect portions; in group 4, the simple striped fibrous protein/chitosan micropattern conduits were transplanted in sciatic nerve defect portions; and in group 5, EMSCs/striped fibrous protein/chitosan micropattern conduits were transplanted in sciatic nerve defect portions. After conduit transplantation, fibrin glue was used to seal anastomotic stomas and muscles and skins were sutured (see FIGS. 8A˜8C for the operation process). All groups were conventionally bred after operations, and sciatic nerve indexes were regularly measured.

(2) Evaluation Indexes for Effects of Repairing Nerve Injury by Conduit Transplantation

1) Observation of General Conditions and Measurement of Sciatic Functional Index (SFI)

The diet, foot ulcer, limb activity and incision healing conditions of the rats were observed conventionally after the operations. The SFI was measured each week: a two-end-open wood trough which is 60 cm long, 10 cm wide and 20 cm high was manufactured, and 70 g/m² white paper was cut to be equal to the wood trough in length and width and then laid at the bottom of the trough. Bilateral hind limbs of each rat were dipped in pigment to color double ankle joints, the rat was placed at one end of the trough and made to walk to the other end of the trough by itself, and 5-6 footprints were left by the hind limb on each side. Three indexes of normal feet (N) and injured feet (E) were respectively measured in selected clear footprints: A, PL (print length), B, TS (toe spread) and C, IT (intermediary toe spread). The above indexes were substituted into a Bain formula, and the SFI was worked out.

SFI=109.5(ETS−NTS)/NTS−38.3(EPL−NPL)/NPL+13.3(EIT−NIT)/NIT−8.8. Bain formula:

SFI=0 means normal, and −100 means complete injury.

2) Fluorochrome Retrograde Tracing

Three rats were randomly selected from each group for fluorochrome retrograde tracing 15 weeks after the operations (1 week before the observation end point). The sciatic nerves were exposed again after anesthesia, and 2 μL of a 5% fluorochrome-phosphate buffer saline (PBS) solution was injected with a microsyringe at the 5 mm position of the far end of a transplant. The same amount of fluorochrome was also injected to the corresponding positions of the sciatic nerves on the normal sides. Operative incisions were sutured, and the animals continued to be bred. Corresponding L4-L6 and S1-S2 dorsal root ganglions on the left and right sides were taken out 1 week later and longitudinally sliced with the thickness of 10 μm by a freezing microtome. Ten consecutive slices were observed respectively under a fluorescence microscope (since ganglions are small, the complete picture can be observed within a lower power field), the total number of positive cells labeled with fluorochrome in each slice was counted by using Image-proPlus6.0, and an average value was calculated. The positive cell ratio was labeled on double sides (positive cell ratio=positive cell number labeled on the experimental side/positive cell number on the control side×100%) and configured to reflect the nerve regeneration degree (the total number of the positive cells is positively correlated to the conduit transplantation repairing effect).

3) Morphological Observation and Measurement of Gastrocnemii

16 weeks after the animal operations, bilateral gastrocnemii were completely cut and weighed with an electronic balance (accurate to 0.001 g), and the wet weight ratio of the bilateral gastrocnemii of the animals in each group was calculated (wet weight ratio=wet weight of experimental side muscle/wet weight of control side muscle×100%). After weighing, the muscle was fixed with a 4% paraformaldehyde solution and embedded in conventional paraffin. The tissue slices were stained with H-E and observed under a light microscope. The cross-sectional area of left and right gastrocnemius fibers was measured by a Leica microscopic image analysis system, and the cross-sectional area ratio (cross-sectional area ratio=cross-sectional area of experimental side muscle/cross-sectional area of control side muscle×100%) was calculated.

4) Morphological Observation and Measurement of Sciatic Nerves

16 weeks after the animal operations, after the animals were anesthetized through the above method, the original incisions were cut open to expose the sciatic nerves, and the regeneration condition of the sciatic nerves was observed. The rats with and without the transplanted nerve conduits after sciatic nerve injury were selected, each sciatic nerve after injury repairing included a near section (upper section), an injured section (conduit transplantation portion) and a far section (lower section) of the injured portion, and after being fixed with a 4% paraformaldehyde solution, the nerves were embedded in conventional paraffin and sliced. The slicing direction was parallel to a nerve longitudinal axis in the longitudinal direction and passed the near section (upper section), the injured section (conduit transplantation portion) and the far section (lower section), so that the condition of the regenerated nerve fibers passing the conduits was conveniently observed. Tissue slices were subjected to H.E staining and immunohistochemical staining with an antibody of the nerve fiber marker protein NF-200 respectively. Microscopic observation and image collection were performed, the density of the nerve fibers was measured by an image analysis system (the cross sections of the thickest portions of the longitudinal sections of the sciatic nerves in each group of animals were selected for comparison).

Results show that a tissue-engineered nerve transplant provided by the disclosure can promote nerve regeneration and recovery of a lower limb motor function through injured portion transplantation.

What is claimed is:

1. A tissue-engineered nerve transplant, comprising a biomaterial that comprises a surface provided with a striped micropattern, the biomaterial is used as a scaffold, and the scaffold is inoculated with seed cells to form the tissue-engineered nerve transplant, and wherein the seed cells comprises ecto-mesenchymal stem cells (EMSCs).

2. The tissue-engineered nerve transplant according to claim 1, wherein the micropattern technology comprises photoetching, electron beam lithography or nanoimprint lithography.

3. The tissue-engineered nerve transplant according to claim 2, wherein the striped micropattern has a width of 1-2 μm, a spacing of 1-2 μm and a stripe height of 1-2 μm.

4. The tissue-engineered nerve transplant according to claim 3, wherein one or more of polydimethylsiloxane, polycaprolactone, chitosan and fibrinogen are used as the biomaterial.

5. The tissue-engineered nerve transplant according to claim 4, wherein the biomaterial comprises chitosan-fibrous protein.

6. The tissue-engineered nerve transplant according to claim 5, wherein the chitosan-fibrous protein is obtained by crosslinking chitosan and fibrinogen with a cell growth factor through a biological crosslinking agent, and the cell growth factor is one or more of epidermal growth factor(EGF), fibroblast growth factor (FGE), nerve growth factor (NGF) and sonic hedgehog homolog (SHH).

7. The tissue-engineered nerve transplant according to claim 6, wherein the biological crosslinking agent comprises genipin and/or glutamine transaminase.

8. The tissue-engineered nerve transplant according to claim 1, wherein an initial cell density of the EMSCs is 10⁴-10⁵ cells/cm².

9. The tissue-engineered nerve transplant according to claim 1, wherein the tissue-engineered nerve transplant is filled with a drug or growth factor sustained release material for promoting nerve growth.

10. A method for preparing the tissue-engineered nerve transplant according to claim 1, comprising the following steps:
(1) preparing a biomaterial scaffold with a micropatterned surface, and performing material-taking culture and amplification of EMSCs; and
(2) planting the EMSCs obtained in step (1) to the micropatterned biomaterial scaffold.

11. A nerve conduit, comprising the tissue-engineered nerve transplant of claim 1, wherein the tissue-engineered nerve transplant is rolled into a single-layer or multi-layer multi-tunnel nerve conduit.

12. The nerve conduit according to claim 11, wherein the striped micropattern has a width of 1-2 μm, a spacing of 1-2 μm and a stripe height of 1-2 μm.

13. The nerve conduit according to claim 11, wherein one or more of polydimethylsiloxane, polycaprolactone, chitosan and fibrinogen are used as the biomaterial.
